(19) United States
(12) Patent Application Publication
(10) Pub. No.: US 2006/0051787 A1
(43) Pub. Date: Mar. 9, 2006

(54) Quantitative PCR for analysis of DNA damage induced by PGHS-2 (COX-2) or other factors

(76) Inventor: Hyesook Kim, Detroit, MI (US)

Correspondence Address: KOHN & ASSOCIATES, PLLC;Suite 410, 30500 Northwestern Highway, Farmington Hills, MI, 48334, US

(21) Appl. No.: 11/169,080

(22) Filed: Jun. 28, 2005

Related U.S. Application Data
(60) Provisional application No. 60/583,927, filed on Jun. 28, 2004.

(51) Int. Cl. C12Q 1/68; C12P 19/34

(52) U.S. Cl. 435/6; 435/91.2

(57) ABSTRACT

A method to assess DNA damage using quantitative polymerase chain reaction (QPCR) with an internal control with or without modified QPCR primers is disclosed. The method further included quantitation of QPCR products using a chemiluminescent, colorimetric or fluorescent assay and a real-time PCR. The chemiluminescent, colorimetric or fluorescent assay is carried out with modified PCR primers and the real-time PCR is carried out with PCR primers with or without modification. A method to select a suitable PCR replication cycle number is disclosed. This is to avoid polymerization of PCR products after addition of the internal control. A method to analyze DNA damage and mutagenicity induced by chemicals in DNA damage repair-deficient and proficient fibroblast cells with endogenous expression of oxidative stress-inducing proteins, e.g., prostaglandin H₂ synthase form 2 (PGHS-2, also called as COX-2), was disclosed.

### CROSS-REFERENCE TO RELATED APPLICATIONS

This application claims the benefit of priority under 35 USC §119 (e) of U.S. Provisional Patent Application Ser. No. 60/583,927, filed on Jun. 28, 2004, which is herein incorporated by reference.

### GOVERNMENT SUPPORT

Research in this application was supported in part by a contract from National Institute of Environmental Health Sciences (R44 ES11251). The government has certain rights in the invention.

### BACKGROUND OF THE INVENTION

1. Field of the Invention

This invention relates to methods to produce cell lines and development of tools to analyze extent of DNA damage induced by PGHS-2 (COX-2) or other factors. More specifically, the invention relates to a method to assess DNA damage using quantitative polymerase chain reaction (QPCR) with an internal control.

2. Description of Related Art

Quan et al. (1) measured the DNA adduct level of ˜0.5 kb nuclear DNA isolated from toxicant-treated cells by quantitative PCR (QPCR). The PCR product level was measured by ³²P-post labeling of the DNA followed by thin-layer chromatography. They found that ˜0.5 kb DNA fragment of an actively transcribed gene (p53 DNA) was susceptible for DNA damage after benzo(a)pyrene-trans-7,8-dihydrodiol (BPD) treatment. Nonetheless, the chance that ˜0.5 kb p53 DNA fragment contained damaged DNA was very low. Thus, other researchers increased the sensitivity of the DNA damage assay by PCR replication of DNA longer than 0.5 kb including 2 kb adenine phosphoribosyltransferase gene (2), 2.6 kb mouse duhydrofolate reductase gene (3), 2.3 kb mitochondrial DNA (3), 16.2 kb mitochondrial DNA (4,5) and 17.7 kb beta-globin gene (4,5). Quantitation of PCR products by the researchers was performed by adding ³²P-labeled nucleotide in the PCR mixture (2-5). No studies were carried out with an internal control DNA added in QPCR mixture to normalize and/or quantitate PCR product levels. Visualization of QPCR products via biotinylated primers which produced single biotin conjugated DNA fragment was previously thought to be too weak to be used for QPCR. However, this method was successfully used for QPCR of 16.2 kb and 8.8 kb DNA fragments as described below. Though it is not as sensitive as QPCR with ³²P-labeled nucleotide in the PCR mixture, this method produced meaningful results as described in the detailed description of the invention.

Real-time QPCR is an analysis of PCR kinetics to detect PCR products as they accumulate. The PCR products are measured by visualizing double stranded DNA or by a 5′nuclease assay which detects a primer bound to its complementary nucleotide sequence and elongated by a polymerase. In the 5′nuclease assay, a PCR primer containing 5′-end reporter dye and 3′-end quencher binds to the complimentary DNA sequence and this primer is elongated by the polymerase. Prior to elongation, the 3′-end quencher is cleaved and diffuses away. Then, the reporter dye emits a fluorescent signal. The level of fluorescence is proportional to the PCR primer successfully incorporated. In the method of detection of double stranded DNA, a DNA intercalator such as ethidium bromide and SYBR® Green I binds to the double stranded DNA. The level of the dye signal is proportional to PCR replication which produces double stranded DNA fragments.

Laws at al. (6) assayed DNA damage of purified genomic DNA mixed with DNA damaging chemicals by QPCR of 17.7 kb β-globin gene followed by real-time PCR analysis of the PCR product levels. Normalization and quantitation of either QPCR or real-time PCR products were not performed using an internal control spiked to the reaction mixture. It is imperative to normalize the PCR products by an internal control being replicated simultaneously with the target DNA in the reaction mixture to compensate for pipetting errors and location of a tube in a PCR machine.

QPCR to detect DNA damage was carried out in the present invention with an internal control DNA produced by shortening a target DNA with intact PCR primer binding sites. Alternatively, DNA damage was analyzed by QPCR with a set of primers which produced two PCR products (long and short) and the short fragment with minimal DNA damage served as an internal control.

Simian virus 40 (SV40)-transformed fibroblasts were treated with 200 and 400 μM hydrogen peroxide for an hour (4,5). PCR of a 16.2 kb mitochondrial fragment and 17.7 kb fragment of the nuclear beta-globin gene obtained from the cells revealed that the level of PCR products of the mitochondrial fragment rapidly decreased to about 20%-30% of that of the nuclear beta-globin gene. Interestingly, DNA damage caused by treatment with 200 μM H₂O₂ for 15 minutes was completely repaired in 1.5 hours in both the mitochondrial DNA and the nuclear gene, while 60 minutes treatment of the cells with the same amount of H₂O₂ showed complete DNA repair of the nuclear gene but no repair in mitochondrial DNA in 3.5 hours.

A DNA damage repair-deficient, xeroderma pigmentosum group A (XPA) human skin fibroblast cell line which expressed cytochrome P450 1A1 showed approximately three-fold higher mutation frequencies than those of a wild type cell line by an endogenously formed metabolite of benzo(a)pyrene-7,8-dihydrodiol (BPD) (7). Whereas the BPD is a substrate for cytochrome P450 1A1, it is not a substrate for PGHS. Previously, arachidonic acid (AA), which is a substrate of a partial enzyme of PGHS with PGG₂ formation (cyclooxygenase) activity, was thought to be necessary to couple BPD biotransformation. However, applicant found no DNA damage was induced with AA. It was necessary to co-treat t-BOOH which is a substrate for the peroxidase of PGHS to induce DNA damage by BPD.

In the prior art, human PGHS-2 was expressed in a baculovirus/insect cell system which has been a most popular PGHS-2 expression system. A majority of COX-2 drug screening has been carried out with this system. However, the arachidonic acid metabolite profile of the PGHS-2 expressed in the insect cells differs from that of native PGHS-2, as shown in the summary of the present invention below.

### SUMMARY OF THE INVENTION

A method to assess DNA damage using quantitative polymerase chain reaction (QPCR) with an internal control with or without modified QPCR primers is disclosed. The method further included quantitation of QPCR products using a chemiluminescent, colorimetric or fluorescent assay and a real-time PCR. The chemiluminescent, colorimetric or fluorescent assay is carried out with modified PCR primers and the real-time PCR is carried out with PCR primers with or without modification. A method to select a suitable PCR replication cycle number is disclosed. This is to avoid polymerization of PCR products after addition of the internal control. A method to analyze DNA damage induced by chemicals in DNA damage repair-deficient and proficient fibroblast cells with and without endogenous expression of oxidative stress-inducing proteins, e.g., prostaglandin H₂ synthase form 2 (PGHS-2, also called as cyclooxygenase, COX-2), was disclosed.

### DESCRIPTION OF THE DRAWINGS

Other advantages of the present invention will be readily appreciated as the same becomes better understood by reference to the following detailed description when considered in connection with the accompanying drawings, wherein:

FIG. 1 shows prostaglandin H₂ synthase (PGHS, also called as cyclooxygenase, COX) form 2-dependent hypoxanthine phosphorybosyltransferase⁻ (HPRT⁻) mutants. Induction of 6-thioguanine resistance was assayed for mutagenicity. A DNA damage repair-deficient, xeroderma pigmentosum group A (XPA) human skin fibroblast cell line, with (Cell #12, PGHS-2⁺) and without (Cell #32, PGHS-2⁻, negative control XPA) transfected human PGHS-2 gene, were preincubated in 20 μM hypoxanthine, 20 μM thymidine, 20 μM glycine, and 1.6 μM aminopterin to reduce background levels of HPRT⁻. After 24 hours, 1.0×10⁶ cells were replated in 10 cm dish in 7 ml of medium without hypoxanthine, glycine, or aminopterin but with 5 μM thymine. After 16 hours, the cells were treated with 0.05 mM t-BOOH or 0.05 mM t-BOOH+10 μM BPD at 37° C. for 3 hours. The cells were incubated for approximately 10 to 15 days and replated for 6-thioguanine selection of mutants in complete medium containing 7 μg/ml 6-thioguanine per 1.5×10⁵ cells. The mutant colonies were fixed, stained and counted.

FIG. 2 shows genotoxicity assays of DNA damage repair deficient XPA cells #12 after treatment with various chemicals by QPCR of a 16.2 kb mitochondrial DNA fragment.

Panel A, ethidium bromide staining of biotinylated mitochondrial DNA fragments (16.2 kb), Panel B, visualization of the biotinylated mitochondrial DNA fragments by chemiluminescent assay (ECL) system, Panel C, a negative control, ethidium bromide staining of biotinylated β-globin (0.55 kb) DNA fragments and Panel D, visualization of the biotinylated β-globin DNA fragments by an ECL system.

Lane 1, bp standards, lanes 2 and 3, cells treated with 1 mM t-BOOH+solvent for BPD (tetrahydrofuran), lanes 4 and 5, cells treated with 1 mM t-BOOH+5 μM BPD, lanes 6 and 7, cells treated with solvent for AA (ethanol), lanes 8 and 9, cells treated with 100 μM AA and lanes 10 and 11, cells treated with 100 μM AA+5 μM BPD. Cells were treated for 3 hours at 37° C., DNA was isolated and QPCRs were carried out using biotinylated nucleotide primers. The DNA product was separated by 1% agarose gel electrophoresis and visualized by ethidium bromide staining. The DNA fragments were blotted to a nitrocellulose membrane and visualized by a streptavidin-horseradish peroxidase or alkaline phosphatase system with a colorimetric or ECL substrate.

FIG. 3 shows a scheme which explains use of an internal control DNA with intact primer binding sites for normalization and quantitation of 8.8 kb PCR products in DNA damage analysis. A, DNA without damage and B, damaged DNA.

FIG. 4 shows long QPCR DNA replication with biotinylated PCR primers followed by visualization of PCR products with chemiluminescent method. The 6.7 kb internal control containing intact primer binding sites was produced by deleting a 2.1 kb fragment from the 8.8 kb mitochondrial DNA fragment.

XPA cells were treated for 3 hours at 37° C. with t-BOOH (a substrate for peroxidase of PGHS)+benzo(a)pyrene-7,8-dihydrodiol (BPD, premutagen). DNAs from treated and untreated cells were isolated and 14 cycles of PCR of the DNA (15 ng) was carried out using biotinylated nucleotide primers. The DNA product was separated by electrophoresis on 1% agarose gel and visualized by ethidium bromide staining. The DNA fragments were electroblotted to a nitrocellulose membrane and visualized by a streptavidin-horseradish peroxidase system/chemiluminescent substrate. The visualized DNA was quantitated using a laser densitometer with an ImageQuant software (Molecular Dynamics). The 8.8 kb PCR product levels were normalized by dividing with 6.7 kb internal control PCR product levels. The mean value of duplicate samples and difference of the two values is shown as a bar.

FIG. 5 shows long QPCR DNA replication of 16.2 kb and 1.2 kb with one set of biotinylated PCR primers followed by visualization of PCR products with ethidium bromide staining. DNA for QPCR was obtained from DNA damage repair deficient cell line #12. Lane 1, DNA obtained from untreated cells; lane 2, from cells treated with tetrahydrofuran (THF), solvent for benzo(a)pyrene-trans-7,8-dihydrodiol (BPD); lane 3, from cells treated with BPD; from cells treated with 0.5 mM tert-butyl hydroperoxide (t-BOOH); lane 4, cells treated with 0.5 mM t-BOOH+5 μM BPD; and lane 5, from cells treated with 0.5 mM t-BOOH+10 μM BPD. Cells were treated for 3 hours at 37° C., DNA was isolated and 26 cycles of PCR of DNA (15 ng) was carried out using biotinylated nucleotide primers. The DNA product was separated by 1% agarose gel electrophoresis and visualized by ethidium bromide staining.

FIG. 6 shows real-time PCR primer binding sites of 8.8 kb target DNA and 6.7 kb internal control DNA. The 2.1 kb fragment in the middle of 8.8 kb fragment was cut with a Pst1 restriction enzyme and after isolation of 0.9 kb and 5.8 kb fragments, the 6.7 kb fragment was produced by annealing of the two fragments.

FIG. 7 shows cycle-dependent production of 8.8 kb PCR products without polymerization. DNA from XPA cells was isolated and QPCR of DNA (15 ng) was carried out using biotinylated nucleotide primers. The DNA product was separated by electrophoresis on 1% agarose gel and visualized by ethidium bromide staining.

FIG. 8 shows a cycle-dependent polymerization of PCR products. The 6.7 kb internal control containing intact primer binding sites was produced by deleting a 2.1 kb fragment from the 8.8 kb mitochondrial DNA fragment. DNA from XPA cells was isolated and QPCR of DNA (15 ng) was carried out using biotinylated nucleotide primers. The DNA product was separated by electrophoresis on 1% agarose gel and visualized by ethidium bromide staining. The DNA fragments were electroblotted to a nitrocellulose membrane and visualized by a streptavidin-horseradish peroxidase system with a chemiluminescent substrate.

### DETAILED DESCRIPTION OF THE INVENTION

Two problems with DNA damage assays available in the market are as follows: (a) DNA damage is repaired in normal cells, so high level and long treatment of toxicants is necessary; and (b) there are thousands of toxicants and their metabolites which bind to DNA. Thus, the DNA damage assay has to detect any kind of DNA damage. Applicant has developed DNA damage assays to satisfy these needs: a DNA damage assay using (a) DNA damage repair deficient xeroderma pigmentosum group A (XPA) human skin fibroblast cell line to sensitize DNA damage and (b) long QPCR to detect any kinds of DNA damage.

Mutagenicity and genotoxicity with a bacterial test is not suitable because reactive toxicants produced by PGHS-2 are short-lived and cannot easily cross the bacterial membrane. Expression of human PGHS-2 in a bacterial system produces PGHS-2 protein inactive due to lack of glycosylation. Thus, applicant developed PGHS-2 expressing human cell lines: DNA damage repair deficient cells and DNA damage repair proficient cells.

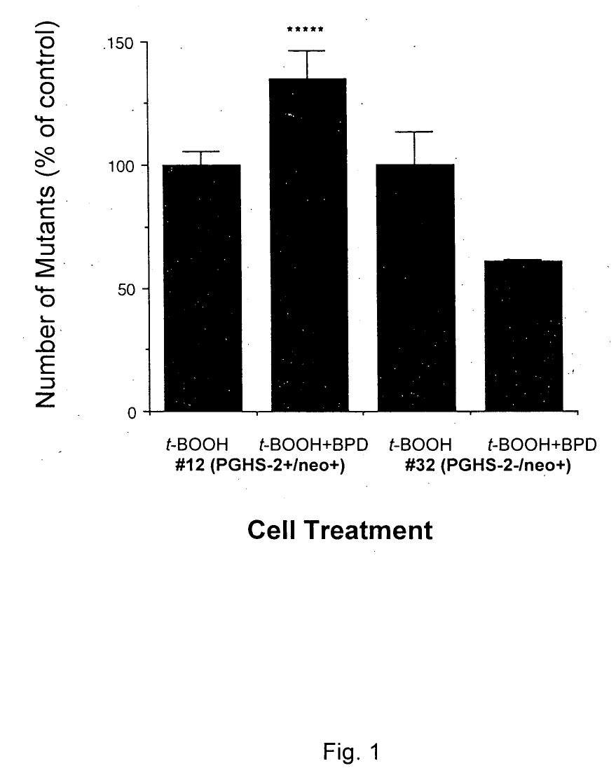

PGHS-2 has two enzymatic activities: PGG₂ formation activity from arachidonic acid (AA) (cyclooxygenase) and PGH₂ formation activity from PGG₂ (peroxidase). Applicant's experiments proved that a pretoxicant, benzo(a)pyrene-trans-7,8-dihydrodiol (BPD) was transformed to a toxicant by PGHS-2 when a substrate of the peroxidase of PGHS-2 (t-BOOH) was changed into t-BOH. The toxicant damaged DNA was evidenced by increased radioactivity of isolated DNA after treatment of the PGHS-2-expressing XPA cells with t-BOOH+[³H]BPD (Table 2) and QPCR assays of 8.8 kb and 16.2 kb mitochondrial DNA after treatment of the XPA cells with t-BOOH+BPD (FIGS. 3 and 4). PGHS-2-dependent hypoxanthine phosphorybosyltransferase⁻ (HPRT⁻) mutation was also observed after treatment of the PGHS-2-expressing XPA cells with t-BOOH+BPD (FIG. 1). Interestingly, treatment of the pretoxicant with AA did not induce any DNA damages. Thus if a PGHS inhibitor (anti-arthritis drug, COX-2 drug) inhibits PGG₂ formation activity of PGHS but does not inhibit peroxidase activity of PGHS, it will inhibit formation of PGE₂ which induces arthritis but toxicity induced by peroxidase activity of PGHS will be still intact. Inhibition of PGG₂ formation activity can increase the peroxidase activity of the PGHS. Considering that arthritis patients have elevated PGHS-2 (COX-2) levels, the toxicity induced by peroxidase of PGHS-2 in the arthritis patients is higher than in a normal person. PGHS-2 levels are also increased in various cancers. An anti-arthritis drug must inhibit PGHS-2 activity but it must not induce oxidative stress which damages DNA. However, a cancer therapy drug must inhibit PGHS-2 activity but it also must damage DNA by inhibiting PGG₂ formation activity of PGHS-2 while keeping peroxidase activity of the PGHS-2 intact or even increased.

This invention includes development of long QPCR DNA damage analysis and production of PGHS-2 expressing DNA damage repair-deficient cells. According to the present invention, DNA damage can be detected by QPCR of DNA and the PCR product levels of the target DNA can be normalized and quantitated using an internal control. The PCR products can be measured by chemiluminescent assay, colorimetric method, fluorescent assay or real-time PCR. A method to select optimal QPCR cycle number is disclosed. Use of DNA damage repair-deficient cells to facilitate detection of DNA damage and mutagenicity is disclosed. Methods to analyze PGHS-dependent DNA damage and mutagenicity as well as selection criteria of drug candidates for arthritis and cancer therapies are disclosed.

PGHS-2-Expressing DNA Damage Repair Deficient Cell Line Production

XPA cells permanently expressing human PGHS-2 were produced by co-transfection of the human PGHS-2 expression vector and pRSV-NEO, a dominant selective marker for antibiotic G418 (geneticin) resistance. Western blot analysis showed two PGHS-2 bands which are a result of a differential glycosylation state of the PGHS-2. Though the calculated molecular weight of PGHS-2 is −67 kDa, glycosylated PGHS-2 is a mixture of 72 and 74 kDa species.

PGHS-2 activity of the selected positive clone #12 was measured with intact cells with treatment of the cells with [¹⁴C]arachidonic acid (AA) (25 μM) (NEN, specific activity 53 mCi/mmol) and PGHS-2 metabolites in the medium were extracted with ethyl acetate and separated by thin layer chromatography (TLC). PGE₂, PGD₂ and PGF_{2α} formed by human PGHS-2 were identified by comparing these metabolites with metabolites formed by sheep PGHS-1. Contrary to PGHS-2 metabolite profile produced with human PGHS-2 expressing insect cells, the PGHS-2 metabolite profile in the human cells was similar to the profile obtained with native human PGHS-2. Densitometric readings of PGE₂ of the TLC at 0, 10, 15, 30, 45 and 60 minute incubation of cells with AA were 83, 865, 1177, 1270, 1141 and 1721 (these are by an arbitrary unit). Addition of indomethacin (final concentration, 0.5 mM), a well known inhibitor of PGHS, inhibited formation of a major band produced by PGHS-2.

PGHS-2-Dependent Cytotoxicity and Mutagenicity

XPA cells #12 (PGHS⁺/neo⁺) and #32 (PGHS⁻/neo⁺) were selected for cytotoxicity and mutagenicity assays. Cytotoxicity assays were carried out by assaying the loss of colony-forming ability as previously described (1).

Cells were treated as follows: (a) no treatment, (b) 100 μM arachidonic acid (AA), (c) 200 μM AA, (d) 5 μM benzo(a)pyrene-7,8-dihydrodiol (BPD), (e) 5 μM BPD+100 μM AA, (f) 5 μM BPD+200 μM AA, (g) 100 μM butylated hydroxyanisole (BHA), (h) 100 μM BHA+5 μM BPD+100 AA and (i) 100 μM BHA+5 μM BPD+200 μM AA. BPD was dissolved in tetrahydrofuran.

Effects of AA, BPD and BHA on PGHS-2 expressing XPA cells #12 and negative control cells #32 are summarized in Table 1. Treatment of XPA cells #32 (PGHS-2⁻/neo⁺), with various chemicals as shown above slightly lowered or failed to change colony number. Treatment of PGHS-2 expressing XPA cells, #12, with AA, BPD or BHA dramatically increased colony number. This result suggests that treatment of PGHS-2 expressing XPA cells for two hours with M and BPD did not produce a cytotoxic molecule.

Induction of 6-thioguanine resistance in XPA cells was assayed for mutagenicity as previously described (1). As expected from results obtained by the cytotoxicity assays of AA+BPD treatment of XPA cells #12, treatment of AA+BPD for two hours failed to induce 6-thioguanine resistance.

To verify lack of genotoxicity induced by PGHS-2 metabolism of AA in presence of BPD, XPA cells #12 (PGHS-2⁺/neo⁺), and XPA cells #32 (PGHS⁻/neo⁺) were treated with 25 μM AA+5 μM [³H]BPD. DNA was isolated and counted for radioactivity. A negligible amount of [³H]BPD was bound to the genomic DNAs in both cell types (Table 2).

However, treatment of XPA cells #12 with 1 mM tert-butyl hydroperoxide (t-BOOH)+5 μM [³H]BPD and 1 mM t-BOOH+5 μM [³H]BPD showed T-BOOH dose-dependent DNA adduct formation (Table 2).

Mutagenicity assays with XPA cells #12 (PGHS-2⁺) and #32 (PGHS-2⁻) were carried out as previously described (1). Number of HPRT⁻ mutants with 6-thioguanine resistance was higher in PGHS-2-expressing XPA cells after treatment with t-BOOH+BPD compared with t-BOOH treatment (FIG. 1). However, in XPA cells #32 (PGHS-2⁻), the mutant number decreased after treatment with t-BOOH+BPD.

Difference of mutant numbers between XPA cells #12 and #32 after treatment with t-BOOH+BPD was statistically significant (p<0.001 shown as ***** in FIG. 1). These results demonstrated that BPD is a PGHS-2-dependent promutagen when hydroperoxide is metabolized by PGHS-2.

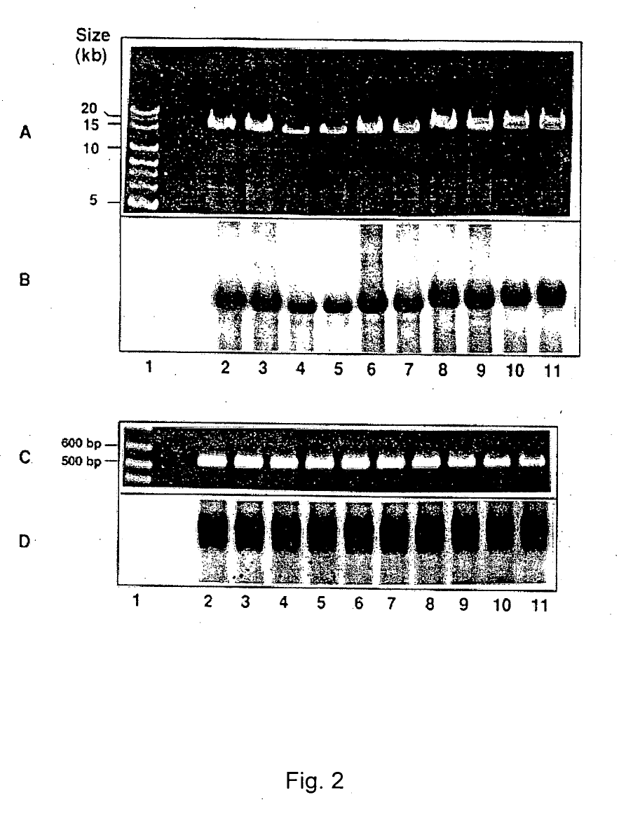

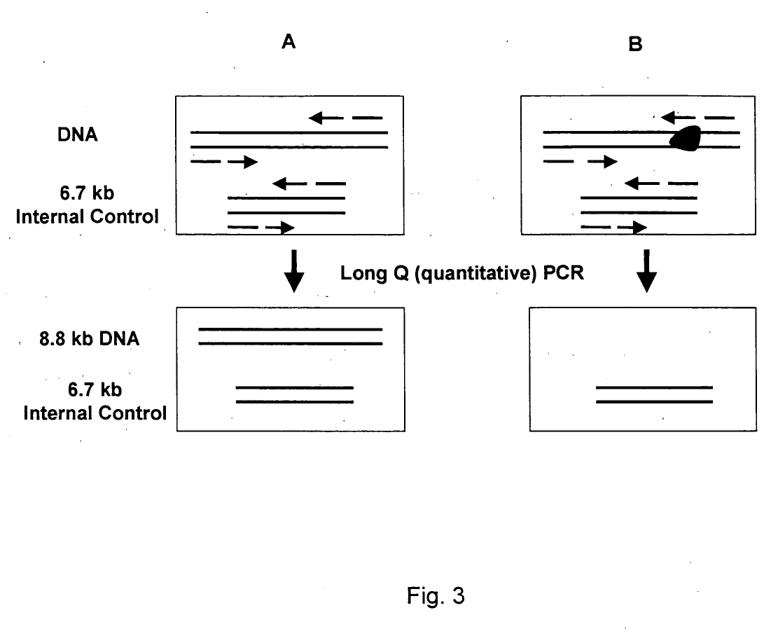

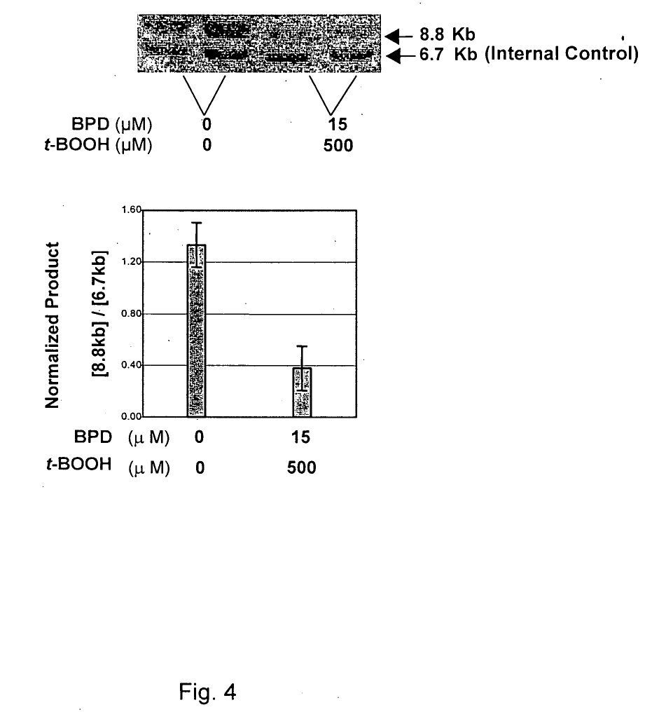

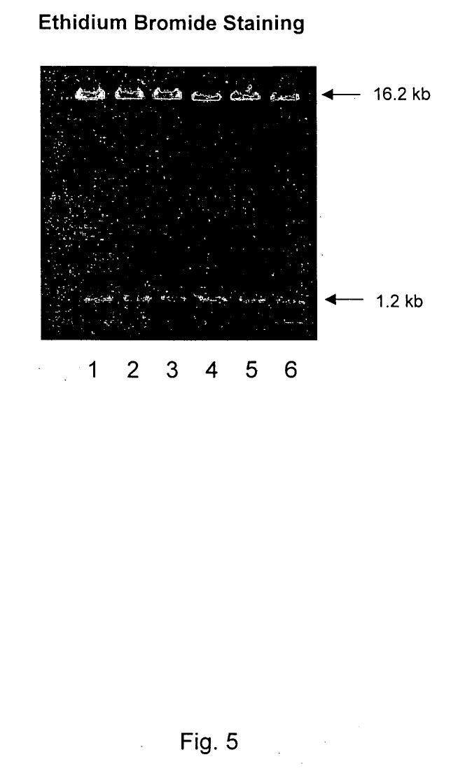

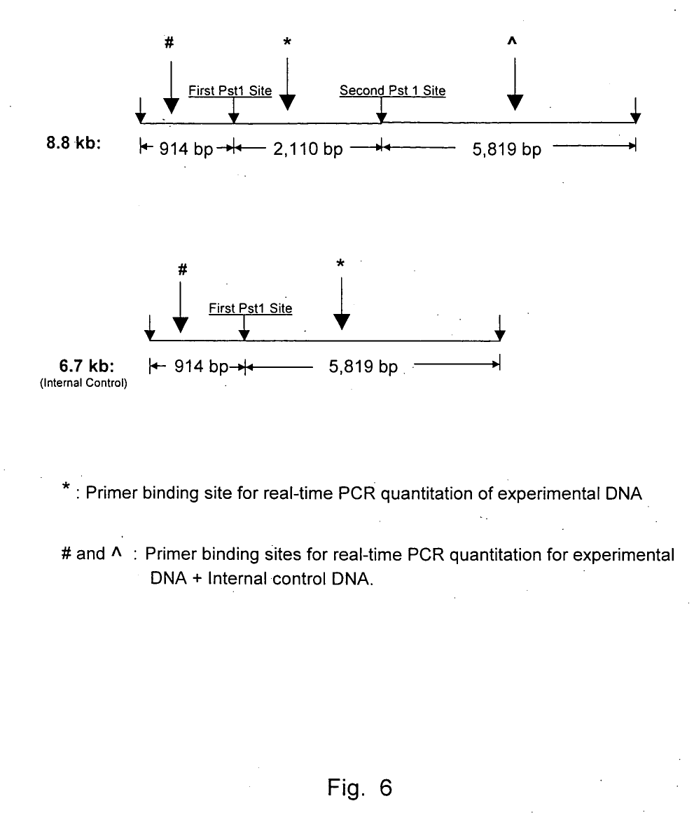

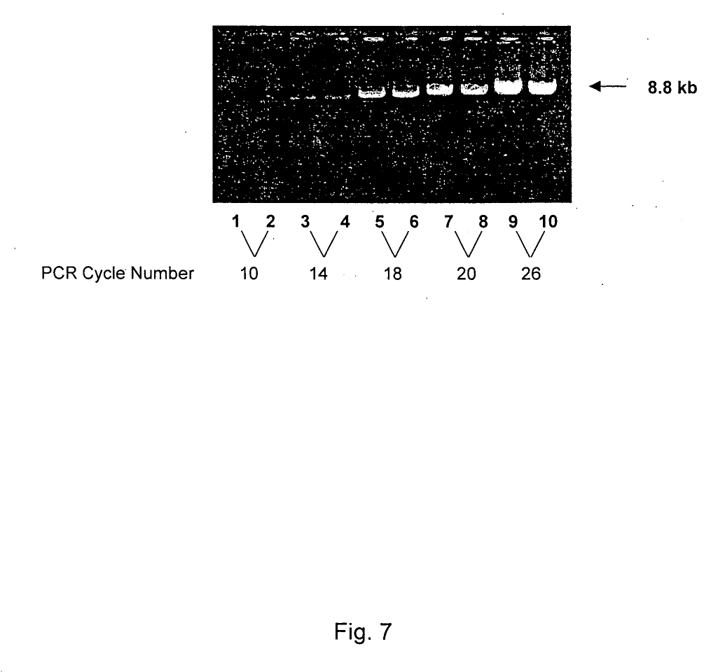

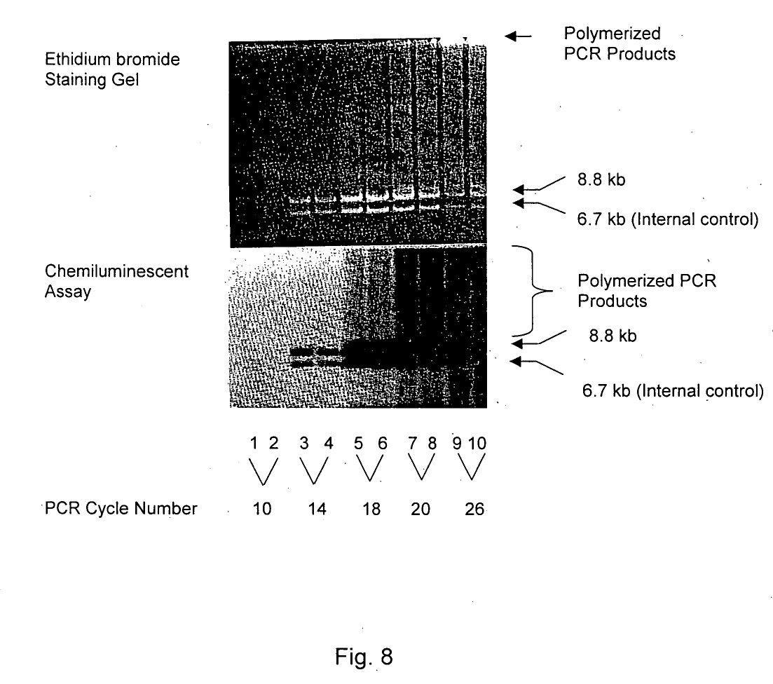

DNA damage analysis of the XPA with QPCR of 16.2 kb mitochondrial DNA and HPRT⁻ mutation analysis revealed that treatment of the cells with AA+BPD did not damage DNA or increase the mutation rate whereas treatment of the cells with t-BOOH+BPD decreased QPCR product level ˜50% (FIG. 2) and increased the mutation rate ˜30% (FIG. 1). This result revealed that QPCR DNA damage assay is a facile and reliable method to predict mutagenicity of a chemical. The mutagenicity assay and the DNA damage assay can be used in combination or each alone.

QPCR DNA Damage Analysis and Normalization/Quantitation of Long QPCR Products Using an Internal Control DNA

PCR technology has been used to replicate DNA. It also can be used to detect DNA damage which prevents DNA replication by DNA polymerase. The principle of QPCR DNA damage analysis is schematically shown in FIG. 3.

DNA damage repair deficient XPA cells #12 (PGHS-2+) were grown and cells were treated for three hours at 37° C. with 0.5 mM or 1 mM t-BOOH with or without 5 μM to 15 μM BPD. DNA was isolated and QPCR of 8.8 kb (FIG. 4) or 16.2 kb (FIG. 2) mitochondrial DNA fragment was carried out using biotinylated nucleotide primers. The PCR product was separated by electrophoresis on 1% agarose gel and visualized by ethidium bromide staining. DNA fragments were transferred to a nitrocellulose membrane and visualized by a streptavidin-horseradish peroxidase/chemiluminescent (ECL) system followed by quantitation with a densitometer. Visualization of the DNA fragments by the ECL system amplified intensity of each DNA band and made it possible to quantitate each DNA band using a densitometer.

For DNA damage analysis using 8.8 kb mitochondrial DNA replication (target DNA), the 6.7 kb internal control DNA with intact primer binding sites was added to the reaction mixture and the 6.7 kb internal control DNA PCR product level was used to normalize and quantitate the 8.8 kb target DNA PCR product levels (FIGS. 3 and 4). The internal control DNA is shorter than the target DNA and also includes the same PCR primer binding site nucleotide sequences as the target DNA. A ratio of the PCR products of the target DNA to the internal control DNA can be used to quantify the original level of the target DNA by comparing the ratio to the known amount of the internal control DNA added to the reaction mixture.

Addition of the internal control DNA to each PCR reaction is necessary to normalize PCR product levels for any experimental variations including pipetting error and location of the tubes in the PCR machine. Previously, a control PCR was carried out with a set of PCR primers different from the target DNA and the small internal control DNA were not replicated in the same reaction mixture. Use of this internal control was not to normalize PCR product levels for any experimental variations including pipetting error and location of the tubes in the PCR machine but for estimation of the copy number of mitochondrial DNA in the sample (8).

PCR products of the 8.8 kb target DNA and 6.7 kb internal control visualized by the ECL method is shown in FIG. 4. When PGHS-2-expressing XPA cells were treated with t-BOOH (PGHS-2 substrate) and BPD (a premutagen), 8.8 kb PCR product level of DNA obtained from these cells dramatically decreased because the DNA was damaged by the oxidized BPD formed by PGHS-2. The 8.8 kb target DNA PCR product levels obtained from two control samples (first and second lanes from left) in FIG. 4 (Western blot analysis) differed approximately two-fold. However, the difference became smaller after the 8.8 kb PCR product levels were normalized using their internal controls (6.7 kb PCR product).

QPCR replication of 16.2 kb DNA with biotinylated PCR primers was carried out with DNA samples obtained from PGHS-2-expressing XPA cells after treatment of the cells with 0.5 mM t-BOOH and 5 μM or 10 μM BPD. The PCR products were separated by agarose gel electrophoresis and visualized by ethidium bromide staining (FIG. 5).

As shown in FIG. 5, 16.2 kb and 1.2 kb PCR products were obtained. It was unexpected that PCR replication of DNA with primers targeted only for 16.2 kb mitochondrial DNA produced the additional 1.2 kb fragment. The 16.2 kb PCR product levels decreased after treatment of cells with 0.5 mM t-BOOH (−50% of control), 0.5 mM t-BOOH+5 μM BPD (˜50% of control) or 0.5 mM t-BOOH+10 μM BPD (˜30% of control) whereas the 1.2 kb PCR products failed to change. Thus, the level of 1.2 kb PCR product can be used for normalization of the 16.2 kb target DNA. Sequence of the 1.2 kb fragment matched with 5′-end of the 16.2 kb DNA sequence.

DNA damage analysis can also be carried out by amplification of a target DNA and a shorter DNA which is a part of the target DNA sequence or a DNA sequence which is not a part of the target DNA by addition of more than one forward and one reverse PCR primers in a reaction mixture to produce two PCR products with different sizes.

The full-length PCR product levels are normalized using the partial PCR product. Alternatively, PCR replication length-dependent DNA damage rate can be calculated and used to obtain differential DNA damage rates of control and experimental samples.

Compared to multiple signal incorporation with [³²P]nucleotide labeling and double stranded DNA binding dye labeling, biotinylated primers used for QPCR incorporate single biotin molecule to each DNA fragment. Thus, it was surprising that the labeling via the biotinylated primers rendered a strong signal with ECL (FIG. 2) and with much insensitive calorimetric alkaline phosphatase (data not shown) systems.

QPCR amplification of the 8.8 kb DNA fragment with 26 cycles, resulted in saturation of PCR product formation activity. This problem solved by lowering PCR amplification cycle number lower than 18 cycles which still produced enough copies of biotinylated PCR products to be easily detected by ECL method.

If NTP mixture containing biotin-14-dCTP is used for QPCR, a very strong signal can be produced. However, this method cannot distinguish a target DNA-dependent signal from the internal control-dependent signal. Two sets of QPCR primers (one set of biotinylated primers for a target DNA and the other conjugated with digoxigenin for an internal control DNA) can be added to the reaction mixture to obtain for each primer set-specific signal. This method can distinguish the target DNA-dependent signal from the internal control-dependent signal.

Quantitation of PCR Products by Real-Time PCR

QPCR products of 2.3 kb and 8.8 kb mitochondrial DNA were quantitated by real-time PCR with primers to produce a 112 bp fragment using the ABI PRISM® 7700 (Applied Biosystems). DNA replication was monitored by addition of a double-stranded DNA binding dye, SYBR® Green I (Applied Biosystems).

As shown in FIG. 6, the real-time PCR primers were produced to bind the 8.8 kb target DNA fragment but not to the 6.7 kb internal control. Real-time PCR primers selected from the 6.7 kb internal control DNA sequence bind both 8.8 kb and 6.7 kb fragments.

Concentrations of 8.8 kb PCR products were obtained after treatment of PGHS-2-expressing DNA damage repair deficient cells with t-BOOH (PGHS-2 substrate) and BPD (a premutagen) using a standard curve. The standard curve was obtained by real-time PCR with various concentrations of the 8.8 kb DNA.

The PCR product level of control was ˜112 pg. However, after treatment of cells with 0.5 mM t-BOOH+10 μM BPD, the PCR product level became ˜50 pg (˜45% of control) due to DNA damage induced by BPD after biotransformation to DNA binding molecules by PGHS-2 (Table 3).

PCR product levels of the target and internal control DNA can be obtained by (a) measurement of total (target and internal control DNA) and target DNA PCR products and (b) subtracting target DNA PCR levels from total to obtain internal control DNA PCR product level. Each target DNA PCR product level can be normalized by dividing with its internal control level.

QPCR Cycle-Dependent Polymerization of PCR Product

PCR cycle-dependent 8.8 kb fragment production was studied after replication of 15 ng of DNA by 10, 14, 18, 22 and 26 PCR cycles. The 8.8 kb PCR product level was PCR cycle dependent. A single strong band formed up to 26 PCR cycle (FIG. 7). However, when a 6.7 kb internal control was added to the PCR reaction mixture containing one set of PCR primers for both 8.8 kb target DNA and 6.7 kb internal control (FIG. 3), QPCR procedure produced polymerized PCR products at PCR cycle number higher than 18. The formation of the polymerized product was PCR cycle dependent (FIG. 8). It was a surprise that the PCR products were polymerized when the internal control DNA was added. This result suggests that QPCR cycle number has to be experimentally selected for a suitable DNA damage assay development.

The above discussion provides a factual basis for the method of the present invention for production and use of antibody and/or focused (non-global) microarray. The methods used with and the utility of the present invention can be shown by the following non-limiting examples and accompanying figures.

### EXAMPLES

Materials and Methods

Materials

[¹⁴C]arachidonic acid was obtained from New England Nuclear (NEN). An alkaline phosphatase or horseradish peroxidase system and their chemiluminescent substrates were from Amersham. Benzo(a)pyrene-7,8-dihydrodiol (BPD) and [³H]BPD were obtained from NCI Chemical Carcinogen Repository (Midwest Research Institute, Kansas City Mo.). Antibody for PGHS-2 was from Cayman Co. Platinum Pfx DNA polymerase was obtained from Life Technologies. rTth DNA polymerase was from Roche Molecular Systems. Biotinylated and non-biotinylated PCR primers were synthesized by Invitrogen. Other reagents were obtained from Sigma Chemical Co.

PGHS Activity Assay:

PGHS activity was measured after incubation of cells with [¹⁴C]arachidonic acid (AA). The cells were scraped with rubber policeman and resuspended in 0.2 ml of phosphate-buffered saline (PBS), [¹⁴C]AA (25 μM) (NEN, specific activity 53 mCi/mmol) was added to the cell suspension and incubated at 37° C. up to 60 minutes. PGHS-2 metabolites in the medium were extracted with ethyl acetate, dried down in a centrifugal evaporator, spotted on a silica thin layer chromatography (TLC) plate and separated using A-9 solvent (ethyl acetate:trimethylpentane:acetic acid:water, 55:25:10:50). Quantitation of PGE₂ of the TLC plate was performed using a phosphorimager (BioRad). The TLC assay measures products resulting from both the cyclooxygenase (PGG₂ formation activity) and peroxidase activities of PGHS. The immediate product of the PGHS reaction, PGH₂, is unstable and breaks down in a non-enzymatic fashion to a variety of prostaglandins which are detectable by TLC. The R_{f} pattern in the A-9 solvent system of the breakdown products of PGH₂ produced by ram seminal vesicle PGHS-1 is well characterized. Thus, PGE₂, PGD₂ and PGF_{2α} formed by human PGHS were identified by comparing these metabolites with metabolites formed by the sheep PGHS-1.

Cell Growth and Chemical Treatment

DNA damage repair deficient or proficient cells were grown in minimal essential medium with α-modification (α-MEM) supplemented with 10% fetal bovine serum, geneticin (50 μg/ml, Gibco-BRL) and 10 mM HEPES, pH 7.0, in a humidified incubator at 37° C. with a 5% CO₂ atmosphere. Cells were treated at 37° C. with or without addition of AA, t-BOOH and/or various concentrations of benzo(a)pyrene-7,8-dihydrodiol (BPD).

Mutagenicity Assays

Induction of 6-thioguanine resistance in XPA cells was assayed for mutagenicity as previously described (1). Briefly, cells were preincubated in 20 μM hypoxanthine, 20 μM thymidine, 20 μM glycine, and 1.6 μM aminopterin to reduce background levels of HPRT⁻. After 24 hr, 1.0×10⁶ cells were replated in 10 cm dish in 7 ml of medium without hypoxanthine, glycine, or aminopterin but with 5 μM thymine. After 16 hr, the cells were treated with 100 μM AA+5 μM BPD or 50 μM t-BOOH+10 μM BPD for 2-3 hours. The cells were incubated for ˜10 to 15 days and replated for 6-thioguanine selection of mutants in complete medium containing 7 μg/ml 6-thioguanine per 1.5×10⁵ cells. The mutant colonies were fixed, stained and counted.

Quantitative PCR (QPCR)

QPCR of a 16.2 kb, 8.8 kb and 2.3 kb mitochondrial DNA and 2.3 kb p53 and 0.5 kb β-globin nuclear DNA were carried out using biotinylated nucleotide primers. The reaction mixture contained 15 ng of template DNA, 1.2 mM MgSO₄, 0.2 mM deoxynucleotide triphosphates, 0.2 μM biotinylated primers, and 1 unit of Platinum Pfx DNA polymerase (Life Technologies). Alternatively, the QPCR was carried out with the GeneAmp XL PCR kit (PerkinElmer/Roche) which contains rTth DNA polymerase XL and XL PCR buffer as previously described (9).

The 6.7 kb internal control DNA produced by deleting 2.1 kb DNA fragment from the 8.8 kb DNA as shown in FIG. 6 was added to the reaction mixture to produce the PCR product level of the 6.7 kb fragment to be a similar level of the 8.8 kb fragment obtained from a control sample (FIG. 4).

The PCR was initiated with a 90° C. hot-start addition of the polymerase. Initial denaturation was carried out for 1 min at 94° C. followed by cycles of denaturation at 94° C. for 15 sec, primer extension and annealing at 66° C. for 12 min and final extension at 72° C. for 10 minutes. The DNA product was separated by electrophoresis on 1% agarose gel and visualized by ethidium bromide staining.

Visualization of PCR Products with Chemiluminescent Assays

DNA fragments separated by electrophoresis on 1% agarose gel were depurinated, denatured and transferred to a nitrocellulose membrane and visualized by a streptavidin-horseradish peroxidase/ECL system. DNA fragments visualized by an ECL system were quantitated using a laser densitometer with an ImageQuant software (Molecular Dynamics, Sunnyvale, Calif.). The target DNA level of treated sample was normalized by dividing the internal control DNA level. Alternatively, the PCR products were visualized by a colorimetric assay with a streptavidin-alkaline phosphatase system.

Quantitation of PCR Products with Real-Time PCR

Best fitting real-time PCR primers were selected using Primer Express program (Applied Biosystems). The PCR primers selected for a real-time PCR were used to replicate 112 bp of PCR for both 8.8 kb and 2.3 kb mitochondrial DNA. The primer binding sites are located in the middle 2.1 kb section (the missing part in 6.7 kb internal control) of 8.8 kb fragment. Thus, the 6.7 internal control did not contribute to 2.4 kb or 8.8 kb mitochondrial DNA quantitation by real-time PCR. The real-time PCR primers for 112 bp product (Tm, 79° C.) are CCACCCTACCACACACATTCGAA (forward primer, nucleotide sequence No. 7401-7421, 21-mer) and AAAAGTCATGGAGGCCATGG (reverse primer, nucleotide sequence No. 7493-7512, 20-mer).

Real-time QPCR was carried out with the SYBR® Green I (a double-stranded DNA binding dye) using the ABI PRISM™7700 (Applied Biosystems).

Standard curve for real-time PCR analysis was obtained with the 8.8 kb standards ranging from 0.012 to 1200 pg. Concentrations of the 8.8 kb DNA preparation were spectrophotometrically measured. PCR was carried out with 2 min pre-incubation at 50° C. and 10 min pre-denaturation at 95° C. followed by 40 cycles of 15 sec at 95° C. and 1 min at 60° C. A melting curve was obtained by dissociation for 20 min from 60° C. to 95° C. Replication cycle number to reach the threshold (Ct) at 0.2 was reciprocally related to concentration of standards. Using the standard curve to obtain relationship of log concentrations of various standards and Ct, QPCR product levels of the samples were calculated.

### EXAMPLE 1

Production of PGHS-Expressing Cell Lines

The well-established Salmonella mutagen test system is not suitable for PGHS because expression of the membrane-bound, glycosylated PGHS in bacterial system has not been successful. Mutagenicity and genotoxicity assays have to be carried out with PGHS-2 expressed in eukaryotic cells.

Thus, human PGHS-2-expressing XPA cells (PGHS-2⁺/neo⁺), human PGHS-2-expressing wild type cells (PGHS-2⁺/neo⁺) and corresponding control cell lines (PGHS-2⁻/neo⁺) were produced.

Selection of XPA and wild type fibroblast cells expressing PGHS (˜70 kDa) were carried out by Western blot analysis using anti-PGHS-2 IgG. PGHS activity of the selected positive cells was measured with intact cells by TLC. Formation of PGE₂, PGD₂ and PGF_{2α} by PGHS-2 was time-dependent.

### EXAMPLE 2

Cytotoxicity, Genotoxicity and Mutagenicity Assays of Environmental Toxicants Using PGHS-2-Expressing DNA Damage Repair-Deficient Cells (GM4429)

Cytotoxicity, genotoxicity and mutagenicity assays to detect the PGHS-2-dependent transformation of promutagens to mutagens has been developed using PGHS-2 expressing DNA damage repair-deficient cells (GM4429). Use of endogenous PGHS-2 for the mutagenicity assay has some advantages. Extracellularly generated, unstable or reactive mutagenic intermediates (free radical metabolites) may not be detected using a conventional bacterial mutagenicity system. A more serious problem with an exogenous PGHS system is the severe toxicity in mammalian testing systems.

Cytotoxicity of environmental toxicants to PGHS-2⁺/neo⁺ (PGHS-2-dependent) XPA and wild type cells were assayed by measuring colony-forming ability. Mutagenicity of the chemical was assayed by measuring induction of 6-thioguanine resistance of the cells, which was a result of mutation of the cells to the HPRT-phenotype.

Cytotoxicity (Table 1) and mutagenicity (FIG. 1) assays were carried out in XPA cells with (cell #12) and without (cell #32) endogenous PGHS-2 using BPD, a known promutagen metabolized by PGHS-2.

Cytotoxicity assays were carried out by assaying the loss of colony-forming ability. Briefly, after harvesting, cells were plated at a cloning density of 600 cells/well in six-well plates and incubated for 7-12 days. The colonies were washed with PBS, fixed with methanol, stained with 0.04% methylene blue, and counted.

It has been reported that BPD was transformed to a mutagen through lipid peroxy radicals which were formed during arachidonic acid (AA) conversion to PGG₂ by cyclooxygenase activity of PGHS-2. Contrary to the previously reported results obtained with exogenous PGHS-2, 5 μM BPD with endogenous PGHS-2 with 100 μM AA in XPA cells, did not induce genotoxicity and cytotoxicity but induced PGHS-2-dependent cell proliferation. As expected, no radioactivity was detected with DNA after cells were treated with AA+[³H]BPD. BPD treatment increased AA-dependent cell proliferation.

PGHS has cyclooxygenase and peroxidase activity. Treatment of XPA cells with 1 mM t-BOOH (a substrate for peroxidase activity of PGHS) and 5 μM [³H]BPD showed PGHS-2-dependent DNA adduct formation as detected by radiolabeled DNA (53.9 pg BPDE/10 μg DNA). Treatment of XPA cells with 1 mM t-BOOH and 5 μM BPD also showed DNA damage with QPCR genotoxicity assay. These results suggested that catalysis of t-BOOH by PGHS-2 produced radicals which oxidated BPD to benzo(a)pyrene-trans-7,8-dihydrodiol-9,10-epoxide (BPDE) to form DNA adducts.

Number of mutant cells resistant to 6-thioguanine treatment (HPRT⁻) increased only with XPA cells #12 (PGHS⁺) (FIG. 1). Difference of mutant numbers between XPA cells #12 and #32 after treatment with t-BOOH+BPD was statistically significant (p<0.001 shown as *****). As expected, the mutation rate did not increase when cells were treated with AA+BPD. These results strongly demonstrated that BPD is a PGHS-2 peroxidase-dependent promutagen.

### EXAMPLE 3

The 16.2 kb, 8.8 kb and 2.3 kb Mitochondrial DNA Damage Analysis Assays

Colorimetric and chemiluminescent DNA damage assays developed with human PGHS-2-expressing XPA cells (Cell #12, PGHS-2⁺/neo⁺) were found to be facile and reliable.

(PGHS-2⁺/neo⁺) was grown in minimal essential medium with α-modification (α-MEM) supplemented with 10% fetal bovine serum, geneticin (50 μg/ml, Gibco-BRL) and 10 mM HEPES, pH 7.0, in a humidified incubator at 37° C. with a 5% CO₂ atmosphere. Cells were treated for 3 hr at 37° C. with or without addition of 0.5 to 1 mM t-BOOH and/or various concentrations of benzo(a)pyrene-7,8-dihydrodiol (BPD).

The colorimetric and chemiluminescent DNA damage assays were developed using biotinylated primers specific for 16.2 kb mitochondrial DNA and 0.55 kb beta-globin gene (a negative control) fragments (FIG. 2).

QPCR products were obtained after 26 PCR cycles. The reaction mixture contained 15 ng of template DNA, 1.2 mM MgSO₄, 0.2 mM deoxynucleotide triphosphates, 0.2 μM biotinylated primers, and 1 unit of Platinum Pfx DNA polymerase (Life Technologies) or rTth DNA polymerase (Roche Molecular Systems). The PCR was initiated with a 90° C. hot-start addition of the polymerase. Initial denaturation was carried out for 1 min at 94° C. followed by cycles of denaturation at 94° C. for 15 sec, primer extension and annealing at 66° C. for 12 min and final extension at 72° C. for 10 minutes. The DNA product was separated by electrophoresis on 1% agarose gel and visualized by ethidium bromide staining. The PCR products were quantitated by streptavidin/horseradish peroxidase system with an ECL substrate.

In addition to this, a colorimetric and chemiluminescent DNA damage assay of 8.8 kb mitochondrial target DNA was developed using a 6.7 kb internal control (FIG. 4). Replication of the 8.8 kb fragment for 14 PCR cycles was selected as a suitable method after a cycle-dependent PCR product polymerization study was carried out (FIG. 8). The PCR products were quantitated by streptavidin/horseradish peroxidase system with an ECL substrate and real-time PCR.

A 2.3 kb mitochondrial DNA was also used for DNA damage analysis after 10 cycles of PCR followed by quantitation of the PCR products with real-time PCR.

### EXAMPLE 4

Internal Control Production

Each QPCR condition may vary due to pipetting variance of PCR ingredients and location of a tube in a PCR machine. Thus, it is imperative to add an internal control in each PCR tube. The internal control has to be smaller than 8.8 kb but intact PCR primer binding sites at both ends of the molecule.

An internal control (6.7 kb) for the QPCR was produced by eliminating an internal 2.1 kb fragment from the 8.8 kb fragment. The 8.8 kb mitochondrial DNA obtained from QPCR of human mitochondrial DNA was cut into 3 pieces at Pst1 sites as shown in FIG. 6. The 0.9 kb and 5.8 kb DNA fragments were purified and annealed. As shown in FIG. 7, 3 fragments with different sizes were produced. They were 5.8 kb (unligated), 6.7 kb (a ligated fragment of 0.9 kb and 5.8 kb) and 11.6 kb (a ligated fragment of 5.8 kb and 5.8 kb). The mitochondrial DNA obtained by the QPCR is 8.843 kb and the internal control is 6.733 kb (FIG. 6).

Cycle-dependent 8.8 kb fragment production by QPCR was studied with addition of 6.7 kb internal control in each tube. QPCR of control mitochondrial DNA (15 ng of template DNA) obtained from XPA Cell #12 and the internal control was carried out using biotinylated nucleotide primers. The 6.7 kb fragment was added and QPCR products were obtained after 10, 14, 18, 20, 22 and 26 PCR cycles. The 8.8 kb and 6.7 kb products were separated by electrophoresis on 1% agarose gel, depurinated, denatured, transferred to a nitrocellulose membrane and visualized by a streptavidin-horseradish peroxidase/ECL system (FIG. 8).

It was a surprise that PCR products were polymerized after PCR replication of the fragments same or longer than 18 cycles. The polymerization of the PCR products were PCR cycle number-dependent. For this specific QPCR condition, QPCR of the samples with 14 cycle was suitable for the DNA damage assays.

PGHS-2-dependent DNA damage by treatment of XPA cells with BPD and t-BOOH was quantitated by QPCR using biotinylated primers specific for both 8.8 kb and 6.7 kb fragments followed by chemiluminescent assays. Results obtained by chemiluminescent assay are shown in FIG. 4. The 8.8 kb and 6.7 kb band intensities were quantitated using a laser densitometer with an ImageQuant software (Molecular Dynamics) and the 8.8 kb band intensity was normalized by 6.7 kb band (internal control) intensity (FIG. 4).

The results demonstrated that levels of 8.8 kb fragments obtained by QPCR decreased after treatment of the cells with BPD with 0.5 mM t-BOOH. Thus, PGHS-2-dependent DNA damage occurred as a result of treatment of XPA cells with BPD and t-BOOH.

When the 16.2 kb mitochondrial DNA was replicated by PCR, a 1.2 kb DNA was also replicated. The PCR amplified 1.2 kb fragment was isolated and the purified 1.2 kb fragment (80 ng) was sequenced using the PCR primers as a sequencing primer. Two DNA sequences, one with a non-biotinylated DNA primer and the other with a biotinylated DNA primer matched the 5′-end of the 16.2 kb mitochondrial DNA sequence. The 1.2 kb PCR product produced without an additional PCR primer set for replication of 16.2 kb DNA fragment served as an internal control (FIG. 5).

### EXAMPLE 5

Real-Time PCR Analysis of PGHS-2-Dependent DNA Damage with benzo(a)pyrene-7,8-dihydrodiol (BPD) and t-BOOH

Contrary to calorimetric and chemiluminescent genotoxicity assays, the real-time PCR requires a specific detection instrument. This approach is a simple, one step procedure which makes it suitable to be used for high-throughput sample analysis.

Real-time PCR analysis was carried out and specificity of the real-time PCR of the 8.8 kb fragment was analyzed by (a) agarose gel electrophoresis of 40 cycle replicated 112 bp real-time PCR product and (b) melting curve of the 112 bp product. A melting curve was obtained by dissociation for 20 min from 60° C. to 95° C. Real-time PCR mixture (50 μl) contained 1.9 μl template from 14 cycles of first PCR, 25 μl SYBR Green solution, 20.8 μl H₂O and 1.1 μl each of 10 μM primers.

Single 112 bp real-time PCR product was detected in each sample and a sharp melting curve at 79° C., which agreeded with the theoretical Tm, was obtained. These results demonstrated that the PCR was specific for the 8.8 kb fragment. The Delta Rn graph was obtained by real-time PCR with 8.8 kb standards and 8.8 kb fragments obtained after 14 cycles of first PCR (2 separate cell culture experiments with 2 separate first PCR and triplicate real-time PCR assays in each cell culture experiment).

Quantitation of 8.8 kb DNA fragments was carried out by measuring PCR cycle number to reach a threshold (0.2). Concentration of 8.8 kb DNA was calculated using a standard curve obtained with known concentrations of 8.9 kb fragments (Table 3). According to the real-time PCR quantitation, it was found that DNA damage occurred after treatment of XPA cells with BPD at 0.5 mM of t-BOOH. In addition, concentrations of 8.8 kb fragments in each sample were quantitated by this method.

The 6.7 kb internal control for QPCR cannot be quantitated by this method because it is produced by deleting a 2.1 kb fragment from the 8.8 kb fragment. However, total DNA (8.8 kb+6.7 kb) levels can be assayed by real-time quantitation with primers which bind both the 8.8 kb and the 6.7 kb.

The 14 cycles were suitable for QPCR replication followed by quantitation of the 8.8 kb fragments using the chemiluminescent assay or real-time PCR.

### EXAMPLE 6

Real-Time PCR Quantitation of 2.3 kb Mitochondrial DNA Fragments

Quantitation of the 2.3 kb QPCR products was carried out by real-time PCR using PCR primers for replication of a 112 bp fragment (same primers were used for quantitation of the 8.8 kb products). The 2.3 kb fragment obtained after 10 cycle QPCR reached the threshold set to 0.2 at −20 PCR cycles. This suggested that the 2.3 kb fragments replicated by 10 cycles of QPCR were suitable for subsequent real-time PCR quantitation.

Real-time PCR replication of the 2.3 kb obtained after treatment of XPA cells with or without various concentrations of BPD and 500 μM t-BOOH was specific as evidenced by a single 112 bp band on an agarose gel electrophoresis obtained after 40 cycle real-time PCR and a sharp peak at 79° C. obtained in a melting curve. Theoretical melting temperature of the 112 bp fragment was 79° C.

Standard curve of the real-time PCR showed a dose-dependent decrease of PCR cycle number to reach the threshold. Untreated samples showed lowest cycle number and BPD and t-BOOH treatment increased the cycle number to reach the threshold.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

TABLE 1
Colony-forming ability of PGHS-2 expressing XPA cells.
Cells were treated with chemicals for two hours, plated
and incubated for 10 days. Colonies were fixed with methanol,
stained with 0.04 methylene blue and counted.

| XPA Cells #12 (PGHS+/neo+) | | XPA Cells #32 (PGHS-/neo+) | |
|---|---|---|---|
| | Colony | | Colony |
| | Number | | Number |
| | (% of | | (% of |
| Treatment | Control) | Treatment | Control) |
| None | 100 | None | 100 |
| 100 μM AA | 133.3 ± 12.6 | 100 μM AA | 70.1 ± 0.6 |
| 200 μM AA | 140.5 ± 8.3 | 200 μM AA | 72.0 ± 6.2 |
| 5 μM BPD | 214.3 ± 7.1 | 5 μM BPD | 88.0 ± 3.3 |
| 100 μM AA + | 251.7 ± 7.6 | 100 μM AA + | 101.1 ± 2.1 |
| 5 μM BPD | | 5 MM BPD | |
| 200 μM AA + | 197.6 ± 46.7 | 200 μM AA + | 85.8 ± 6.1 |
| 5 μM BPD 100 μM | | 5 MM BPD | |
| BHA | 211.9 ± 4.8 | 100 μM BHA | 96.0 ± 3.8 |
| 100 μM BHA + | 181.7 ± 17.1 | 100 μM BHA + | 96.7 ± 11.7 |
| 100 μM AA + | | 100 μM AA + | |
| 5 μM BPD | | 5 μM BPD | |
| 100 μM BHA + | 120.7 ± 16.0 | 100 μM BHA + | 87.5 ± 14.2 |
| 200 μM AA + | | 200 μM AA + | |
| 5 μM BPD | | 5 μM BPD | |

TABLE 2
[³H]benzo(a)pyrene-7,8-dihydrodiol (BPD)-derived DNA adducts
in XPA cells. After three hour treatment, radioactivity of
DNA was assayed by a liquid scintillation counter. AA, arachidonic
acid and t-BOOH, tert-butyl hydroperoxide.

| | BPD Metabolite Bound to DNA (dpm/10 μg DNA) | | |
|---|---|---|---|
| | XPA Cells #12 | XPA Cells #32 | PGHS-2- |
| | (PGHS-2+/ | (PGHS-2-/ | Dependent |
| Treatment | neo+) | neo+) | Adduct Formation |
| Experiment #1 | | | |
| 1 mM t-BOOH + | 78.8 | 33.8 | 45.0 |
| 5 μM [³H]BPD | | | |
| 25 μM AA + | 0 | 0 | 0 |
| 5 μM [³H]BPD | | | |
| Experiment #2 | | | |
| 1 mM t-BOOH + | 197.5 | 130.5 | 67.5 |
| 5 μM [³H]BPD | | | |
| 10 mM t-BOOH + | 232.5 | 75.0 | 167.5 |
| 5 μM [³H]BPD | | | |

TABLE 3
QPCR analysis (14 cycles) followed by real-time
PCR analysis of PGHS-2-dependent DNA damage with benzo(a)
pyrene-7,8-dihydrodiol (BPD) and 0.5 mM of t-BOOH. Cycles
to reach a threshold (0.2) were obtained and DNA
concentration of each sample was calculated by a
standard curve obtained with known concentration□
of 8.8 kb fragments.

| | pg DNA after 14 cycles of first PCR |
|---|---|
| Treatment | (% mean value of control, 112.47 pg) |
| Control 1 | 104.71 ± 5.75 |
| Control 2 | 120.23 ± 1.20 |
| 5 mM BPD 1 | 104.71 ± 3.02 (93.1 ± 2.7%) |
| 5 mM BPD 2 | 125.89 ± 2.59 (111.9 ± 2.3%) |
| 0.5 mM t-BOOH 1 | 79.40 ± 2.24 (70.6 ± 2.0%) |
| 0.5 mM t-BOOH 2 | 52.48 ± 2.19 (46.7 ± 1.9%) |
| 5 mM BPD/0.5 mM t-BOOH 1 | 67.61 ± 1.12 (60.1 ± 1.0%) |
| 5 mM BPD/0.5 mM t-BOOH 2 | 51.29 ± 1.20 (45.6 ± 1.1%) |
| 10 mM BPD/0.5 mM t-BOOH 1 | 45.71 ± 1.10 (40.6 ± 1.0%) |
| 10 mM BPD/0.5 mM t-BOOH 2 | 53.70 ± 1.95 (47.7 ± 1.7%) |
| 15 mM BPD/0.5 mM t-BOOH 1 | 54.95 ± 2.34 (48.9 ± 2.1%) |
| 15 mM BPD/0.5 mM t-BOOH 2 | 54.95 ± 1.66 (48.9 ± 1.5%) |

### LITERATURE CITED

1. Quan, T. and States, J. C. Mol. Carcinogenesis 16:32-43 (1996).
2. Jennerwein, M. M. and Eastman, A. Nucleic acids Res. 19: 6209-6214 (1991).
3. Kalinoswki, D. P., Illenye, S. and Van Houten B. Nucleic acids Res. 20: 3485-3494 (1992).
4. Yakes, F. M. and Van Houten, B. Proc. Natl. Acad. Sci. USA 94:514-519 (1997).
5. Salazar, J. J. and Van Houten, B. Mutat. Res. 385:139-149 (1997).
6. Laws, G. M., Skopek, T. R., Reddy, V., Storer, R. D. and Glaab, W. E. Mutat. Res. 484: 3-18 (2001).
7. Quan, T., Reiners, Jr., J. J., Culp, S. J., Richter, P., and States, J. C. Mol. Carcinogenesis 12:91-102 (1995).
8. Santos J. H., Hunakova, L., Chen, Y., Bortner, C. and Van Houten, B. J. Biol. Chem. 278: 1728-1734 (2003).
9. Ayala-Torres, S., Chen, Y., Svoboda, T., Rosenblatt, J. and Van Houten, B. Methods 22: 135-147 (2000).

1. A method of performing DNA damage analysis, including the steps of:
amplifying DNA with polymerase chain reaction (PCR) primers, wherein the PCR primers are selected to obtain at least 2.3 kb and longer DNA to warrant sensitivity of an assay by adding an internal control DNA shorter than a target DNA, the internal control DNA including PCR primer binding site nucleotide sequences the same as sequences of the target DNA in a reaction mixture;
normalizing DNA levels obtained from said amplification step; and
assessing DNA damage.

2. The method of claim 1, wherein the PCR primers are selected to obtain 8.8 kb and longer DNA.

3. The method of claim 2, wherein the DNA is mitochondrial DNA.

4. The method of claim 3, wherein the internal control DNA is 6.7 kb produced by eliminating a 2.1 kb fragment from a middle section of the 8.8 kb DNA.

5. The method of claim 2, wherein the PCR primers are labeled.

6. The method of claim 5, wherein the PCR primers are biotinylated, and further including after said amplifying step, the step of separating and visualizing the DNA obtained from the amplifying step using streptavidin-reporter conjugates.

7. The method of claim 2, wherein said assessing step is further defined as quantitating the DNA obtained from the amplifying step using real-time PCR with at least one primer set for replication of a piece of DNA sequence present only in the target DNA.

8. The method of claim 7, wherein the DNA is mitochondrial DNA.

9. The method of claim 8, wherein two primer sets in the real-time PCR are used; a first set for replication of a piece of DNA sequence present only in the target DNA and a second set for replication of both the target DNA and the internal control DNA.

10. The method of claim 9, wherein the DNA is mitochondrial DNA.

11. The method of claim 2, wherein the DNA is expressed in DNA damage repair deficient cells.

12. The method of claim 11, wherein the PCR primers are labeled.

13. The method of claim 12, wherein the PCR primers are biotinylated, and further including after said amplifying step, the step of separating and visualizing the DNA obtained from the amplifying step using streptavidin-reporter conjugates.

14. The method of claim 11, wherein said assessing step is further defined as quantitating the DNA obtained from the amplifying step using real-time PCR with at least one primer set for replication of a piece of DNA sequence present only in the target DNA.

15. The method of claim 14, wherein the DNA is mitochondrial DNA.

16. The method of claim 14, wherein two primer sets in the real-time PCR are used: a first set for replication of a piece of DNA sequence present only in the target DNA and a second set for replication of both the target DNA and the internal control DNA.

17. The method of claim 16, wherein the DNA is mitochondrial DNA.

18. The method of claim 11, wherein the DNA is mitochondrial DNA.

19. The method of claim 18, wherein the internal control DNA is 6.7 kb produced by eliminating a 2.1 kb fragment from a middle section of the 8.8 kb DNA.

20. The method of claim 1, wherein the DNA is obtained from prostaglandin H₂ synthase form 2 (PGHS-2)-expressing DNA damage repair deficient cells after co-treatment of a PGHS-2 peroxidase substrate and a chemical.

21. The method of claim 20, wherein the PGHS-2 peroxidase substrate is tert-butyl hydroperoxide (t-BOOH).

22. The method of claim 1, wherein the DNA is obtained from PGHS-2-expressing cells.

23. The method of claim 22, wherein the DNA is obtained from PGHS-2-expressing cells after co-treatment of a PGHS-2 peroxidase substrate and a chemical.

24. The method of claim 23, wherein the PGHS-2 peroxidase substrate is t-BOOH.

25. A method of quantitation of DNA levels, including the steps of:
adding a known amount of internal control DNA to a polymerase chain reaction (PCR) mixture containing a target DNA;
performing a PCR to replicate both the target DNA and the internal control DNA in the PCR mixture to obtain PCR products of the target DNA and internal control DNA; and
obtaining a ratio of PCR products of the target DNA to the internal control DNA to quantify an original target DNA level using the ratio and the known amount of internal control DNA added to the PCR mixture, the internal control DNA being shorter than the target DNA and including PCR primer binding site nucleotide sequences that are the same as sequences of the target DNA in the PCR mixture.

26. The method of claim 25, wherein the DNA is mitochondrial DNA.

27. The method of claim 26, wherein the internal control DNA is 6.7 kb and is produced by eliminating a 2.1 kb fragment from a middle section of an 8.8 kb DNA.

28. The method of claim 25, wherein the PCR includes using labeled PCR primers

29. The method of claim 28, wherein the PCR is carried out with biotinylated PCR primers followed by separation and visualization of the PCR products using streptavidin-reporter conjugates.

30. The method of claim 25, further including the step of quantitating the DNA obtained from the amplifying step using real-time PCR with a primer set for replication of a piece of DNA sequence present only in the target DNA.

31. The method of claim 30, wherein the DNA is mitochondrial DNA.

32. The method of claim 30, wherein two primer sets in the real-time PCR are used: a first set for replication of a piece of DNA sequence present only in the target DNA and a second set for replication of both the target DNA and the internal control DNA.

33. The method of claim 25, wherein the DNA is expressed in DNA damage repair deficient cells.

34. The method of claim 33, wherein the internal control DNA is 6.7 kb produced by eliminating a 2.1 kb fragment from a middle section of 8.8 kb mitochondrial DNA.

35. The method of claim 33, wherein the DNA is obtained from PGHS-2-expressing DNA damage repair deficient cells after co-treatment of a PGHS-2 peroxidase substrate and a chemical.

36. The method of claim 35, wherein the PGHS-2 peroxidase substrate is tert-butyl hydroperoxide (t-BOOH).

37. The method of claim 25, wherein the DNA is obtained from PGHS-2-expressing cells.

38. The method of claim 37, wherein the DNA is obtained from PGHS-2-expressing cells after co-treatment of a PGHS-2 peroxidase substrate and a chemical.

39. The method of claim 38, wherein the PGHS-2 peroxidase substrate is t-BOOH.

40. A method of performing DNA damage analysis, including the steps of:
amplifying a DNA by PCR with lower than 18 PCR cycles to prevent saturation of PCR product formation to increase sensitivity of a DNA damage assay;
assessing DNA damage.

41. A method of performing DNA damage analysis, including the steps of:
amplifying through a polymerase chain reaction (PCR) a target DNA and a shorter DNA in a reaction mixture to produce a long DNA fragment and a short DNA fragment;
normalizing the long DNA fragment using the short DNA fragment; and
assessing DNA damage.

42. The method of claim 41, wherein the PCR is carried out with labeled PCR primers.

43. The method of claim 41, wherein the assessing step is further defined as quantitating the long DNA fragment using real-time PCR with one primer set for replication of a DNA sequence that presents in the long DNA fragment but does not present in the short DNA fragment.

44. The method of claim 43, wherein two primer sets of real-time PCR are used: a first set for replication of a DNA sequence present in the long DNA fragment but not present in the short DNA fragment and a second set for replication of both the long DNA fragment and the short DNA fragment.

45. The method of claim 41, wherein the DNA is obtained from DNA damage repair deficient cells.

46. The method of claim 45, wherein the DNA is obtained from PGHS-2-expressing DNA damage repair deficient cells.

47. The method of claim 41, wherein the DNA is obtained from PGHS-2-expressing cells.

48. A method of analyzing DNA damage, including the steps of:
amplifying DNA with one set of polymerase chain reaction (PCR) primers to obtain two PCR products of a target DNA and an internal control DNA fragment without addition of the internal control DNA in a reaction mixture;
normalizing the target DNA using the internal control DNA fragment;
assessing DNA damage.

49. The method of claim 48, wherein the target DNA is a 16.2 kb mitochondrial DNA and the internal control DNA fragment is a 1.2 kb DNA which is obtained without addition of the 1.2 kb internal control DNA fragment in the reaction mixture.

50. A method to select a suitable polymerase chain reaction (PCR) cycle number for DNA replication with an internal control DNA, including the step of:
identifying a maximal PCR cycle number that does not decrease levels of PCR products of intended sizes due to polymerization of the PCR products.

51. A method of assessing both DNA damage and mutagenicity in a PGHS-2-expressing DNA damage repair deficient cell line by performing a DNA damage assay on DNA from the cell line and by performing a mutagenicity assay on the cell line.

52. A method of assessing DNA damage in a PGHS-2-expressing DNA damage repair deficient cell line by performing a DNA damage assay on DNA from the cell line.

53. A method of assessing mutagenicity in a PGHS-2-expressing DNA damage repair deficient cell line by performing a mutagenicity assay on the cell line.

54. A method of assessing the inhibition of PGHS-2 activity and DNA damage from chemicals, including the step of assessing the inhibition of PGHS-2 activity and DNA damage from chemicals using a PGHS-2-expressing DNA damage repair deficient cell line.

55. The method of claim 54, wherein the assessing step is further defined as using peroxidase activity of PGHS-2 as an index of oxidative stress that induces DNA damage.

56. The method of claim 54, wherein the assessing step is further defined as selecting chemicals that inhibit PGH₂ and PGE₂ formation activity of PGHS-2 but do not damage DNA.

57. The method of claim 54, wherein the assessing step is further defined as selecting chemicals used in a cancer therapy drug that inhibit PGH₂ and PGE₂ formation activity of PGHS-2 but damage DNA.


SEQUENCE LISTING (2 sequences)

SEQ ID NO: 1 - DNA, 23 bp, Artificial sequence
  forward primer
ccaccctaccacacacattcgaa

SEQ ID NO: 2 - DNA, 20 bp, Artificial sequence
  reverse primer
aaaagtcatggaggccatgg